(19) United States
(12) Patent Application Publication
(10) Pub. No.: US 2013/0096390 A1
(43) Pub. Date: Apr. 18, 2013

(54) BioProbe Sheath and Method of Use

(71) Applicants: Laura Weller-Brophy, Pittsford, NY (US); Theodore K. Ricks, Pittsford, NY (US)

(72) Inventors: Laura Weller-Brophy, Pittsford, NY (US); Theodore K. Ricks, Pittsford, NY (US)

(21) Appl. No.: 13/633,787

(22) Filed: Oct. 2, 2012

Related U.S. Application Data
(60) Provisional application No. 61/546,099, filed on Oct. 12, 2011.

(51) Int. Cl. A61B 5/00

(52) U.S. Cl. 600/300

(57) ABSTRACT

This invention relates to a medical device comprising at least one bioprobe for collecting data, a single-use protective cover mechanically attached to said at least one bioprobe, a control module, and a sheath removal system comprising a receiving port for said at least one bioprobe with said attached single-use protective cover, a single-use protective cover locking mechanism for retaining said single-use protective cover, and a single-use protective cover removal feedback mechanism in communication with said control module.

### CROSS REFERENCE TO RELATED APPLICATIONS

Priority is claimed to: U.S. Provisional Patent Application Ser. No. 61546099 by Laura Weller-Brophy and Theodore K. Ricks, entitled “BIOPROBE SHEATH AND METHOD OF USE”, filed on Oct. 12, 2011, the disclosure of which is incorporated herein by reference.

### FIELD OF THE INVENTION

The present invention relates generally to a system for use, removal, and disposal of a medical probe device. More particularly, this invention is directed to a system for controlling the use of a probe sheath or cover used with a medical device, that allows its disposal after use, thereby protecting the user from possible contamination from biohazards that may be retained on the surface of the sheath. The system and method of the present invention also provide a mechanism to prevent sheath reuse, whereby data collected with the probe are not released to the user until the probe sheath is processed by the removal and disposal system.

### BACKGROUND OF THE INVENTION

Spectral measurement systems have been developed for the non-invasive and minimally invasive analysis of a patient's tissues and blood. For example, light-measurement systems are in development, or have been commercialized, to screen for cervical cancer, esophageal cancer, skin cancer, oral cancer, and to measure arterial blockages, plaque build-up, and other medical conditions. The spectral measurement systems commonly include an optical probe that directs light to a tissue surface, wherein light reflected or emitted by the tissue is collected by the optical probe and measured by one or more optical detectors contained within the probe or in a measurement system associated with the probe. The probes can be very slender for use in blood veins and arteries, or can be larger for use in cervical cancer screening and other applications. The probes can be endoscopes that may be of a relatively long length when compared to the diameter of the probe; for example, those probes/endoscopes used in the screening of the colon and esophagus.

Using a spectral measurement system to measure the tissues and blood of numerous patients requires a method or apparatus to render the surface of the probe sterile prior to use with each separate patient. One means of providing a sterile interface between the probe and the patient includes the use of a one-time-use, sterile probe sheath or protective cap.

U.S. Pat. No. 3,809,072, STERILE SHEATH APPARATUS FOR FIBER OPTIC ILLUMINATOR WITH COMPATIBLE LENS, with a May 7, 1974 publication date and incorporated herein, is directed to a disposable sterile sheath for use with an endoscope. The sterile sheath of the invention includes a generally rigid lens element disposed at the tip portion of the sheath. The sheath itself is formed to completely encase the surfaces of the endoscope that contact the patient. The sheath may comprise both the lens element and a tubular sheath body which are sealably connected to each other. The lens element and tubular sheath body may be of the same materials, or may be formed of different materials. Preferably, the tubular sheath body is formed of a flexible material, facilitating the application of the sheath to length of the endoscope and easing the entry of the sheath/endoscope assembly into the body of the patient.

It is of critical importance that a sterile sheath or cap be used. The same spectral imaging apparatus is typically used on a variety of subjects. The sterile sheath or cap reduces the likelihood of spreading contagious diseases from one patient to the next. Without the sterile sheath or cap, the probe could contact biological contaminants including infectious materials in one patient and transmit them to another patient. To avoid the spread of contaminants and infectious materials between patients, it is desired that the sterile sheath or cap be made for a single use, wherein the sheath or cap is disposed of after a single use. In addition, it is important that a means be provided to remove the sheath or cap from the probe, not requiring the end-user to touch the sheath or cap surface, so that the user is not unnecessarily exposed to biological contaminants and infectious materials.

Sheaths and caps for use with the probes of medical measurement systems, including spectral measurement systems, like the sheath disclosed in U.S. Pat. No. 3,809,072, are well known. While most of the sheaths and caps are intended for use with a single patient, it is the user of the probe system who must remember to actually dispose of the sheath or cap after each use, and to replace the sheath or cap with a new, unused, and sterile sheath or cap for each patient. Under certain circumstances, even the most experienced user may forget to dispose of and to replace the probe sheath or cap after use with a patient. The inadvertent exposure of one or more patients to biological contaminants found on the surface of a medical screening device risks the unintended consequences of disease exposure. What is needed, therefore, is a disposable probe sheath or cap that is easy to install and remove and cannot be accidentally reused. In addition, a system is needed that will cause the user of the probe to ensure that a sterile probe sheath or cap is used with each patient.

U.S. Pat. No. 6,549,794, SINGLE USE DISPOSABLE PROTECTIVE CAP, with an Apr. 15, 2003 publication date and incorporated herein, is directed to a disposable protective cap for covering a probe for a spectral imaging apparatus. Features of the disposable protective cap can prevent the cap from being reused. Both mechanical and electrical features may be used alone or together to prevent cap reuse. As noted in the patent summary, “The cap is hollow and elongated to cover the probe, having an open end and a closed end. An optically transparent face is located at the closed end. This permits light to be transmitted from the probe through the closed end of the cap with minimal optical distortion. At the open end, the cap includes components which interact with the probe to assure that the cap is not used more than one time. In some embodiments, these components interact electrically to assure only a single disposable use of the cap. In other embodiments, this is accomplished through mechanical interaction, or a mechanical interaction coupled with an electrical interlocking mechanism. A combination of other electrical and mechanical interactions is used in other embodiments to assure only a single use of the cap.” In specific, the patent teaches methods to prevent reuse of the cap including the incorporation of a “non-reusability unit” located at an open end portion of the cap. The non-reusability unit cooperates with the probe to permit the cap to be operatively mounted to probe only once. One non-reusability unit incorporated in the open end portion of the cap is a one-time use mechanical latch. The one-time-use latch is described as being mechanically disabled so that the latch cannot release and reattach to the probe for a second set of measurements. Use of the one-time-use latch does not, by itself, prevent reuse of the cap, as a user could use the cap in multiple patients. The inventors address this limitation through incorporation of electrical means including electrical interlocking sequences as well as means to identify and track the protective cap use, so that the cap is identified and limited in use to a single patient. Accordingly, the use of the electrical interlocking means and/or cap identification means with the mechanical one-time-use attaching means provide a route to limiting cap reuse. While the patent teaches the use of electrical and mechanical means to prevent reuse of a protective cap or sheath, the invention does not contemplate users having more than one probe system whereby probe sheaths or caps recorded as used by one probe system are not recognized by another system as being used. In this case, a user might reuse a probe sheath or cap because the second probe system would not recognize the probe sheath or cap as having been used. In addition, the invention does not consider users who may find a means to reattach used probe sheaths or caps, even in the case where elements of the sheath or cap are disabled after a first use. The invention fails to describe a means to destroy the cap to prevent reuse, nor does the invention contemplate limiting user access to measured data prior to destruction of the cap. There remains a need for a protective cap and means of use whereby the user is denied access to measured data until the protective cap is rendered incapable of further use, notably through a crushing, ripping, cutting or other means of destroying the physical integrity of the cap.

U.S. Pat. No. 6,847,490, OPTICAL PROBE ACCESSORY DEVICE FOR USE IN VIVO PROCEDURES, with a Jan. 25, 2005 publication date and incorporated herein, is directed to an accessory device (also known as a sheath or cap) to be used with a probe that comprises a body and an attachment element and is mechanically prevented from re-use. In one embodiment, the attachment element attaches the accessory device to the probe and detaches from the body of the accessory device when the accessory device is removed from the probe. The accessory device is unable to function without the attachment element and so detachment of the accessory device from the probe prevents its reuse. The attachment element may comprise a grasping element, such as a tab or a snap ring which detaches the attachment element from the body of the accessory device. In another example, the attachment element is separated from the body of the accessory device by perforations and rupturing the perforations detaches the attachment element from the body of the accessory device.

In yet another example from U.S. Pat. No. 6,847,490, a disposable, single-use accessory device for an optical probe comprises an electrical element rather than a mechanical element which prevents its re-use in another patient. The electrical element may contain encoded information about the accessory device, or may be programmed during use. A system may be provided including a processor and an electrical element reader. The electrical element reader accesses information encoded in the electrical element carried by the accessory device and transmits a signal to the processor relating to identification information carried by the electrical element. The processor includes a memory which stores identification information and which compares the stored information with identification information encoded by the electrical element. In specific, the processor transmits instructions based on whether or not a match is found between identification information encoded in the electrical element and identification information stored within the memory. If no match is found, the identification information encoded in the electrical element is added to the memory. While the patent teaches the use of electrical and mechanical means to prevent reuse of a protective cap or sheath, the invention does not contemplate users having more than one probe system/processor whereby probe sheaths or caps recorded as used by one probe system/processor are not recognized by another system/processor as being used. In this case, a user might reuse a probe sheath or cap because the second probe system/processor would not recognize the probe sheath or cap as having been used. In addition, the invention does not consider users who may find a means to reattach used probe sheaths or caps, even in the case where elements of the sheath or cap are disabled after a first use. The invention fails to describe a means to destroy the cap to prevent reuse. In addition, the invention does not contemplate limiting user access to measured data prior to destruction of the cap. There remains a need for a protective cap and means of use whereby the user is denied access to measured data until the protective cap is removed from the bioprobe and optionally rendered incapable of further use, notably through a crushing, ripping, cutting or other means of destroying the physical integrity of the cap.

US 2002/0117412, DISPENSATION AND DISPOSAL CONTAINER FOR MEDICAL DEVICES, with an Aug. 29, 2002 publication date and incorporated herein, is directed to a container for storage, dispensation, transport and disposal of a medical device. U.S. Pat. No. 6,527,115, DISPENSATION AND DISPOSAL CONTAINER FOR MEDICAL DEVICES, with a Mar. 4, 2003 issue date and incorporated herein, is directed to aspects of a container for storage, dispensation, transport, and disposal of a medical device. US 2003/0132131, METHOD FOR USING DISPENSATION AND DISPOSAL CONTAINER FOR MEDICAL DEVICES, with a Jul. 17, 2003 publication date and incorporated herein, is directed to a method of use of a container for storage, dispensation, transport and disposal of a medical device. More particularly, these inventions are directed to a container and methods for storing a sterile ultrasonic surgical probe that allows its dispensation for use, and for its safe storage and disposal after use, thereby protecting the user from the hazards of accidental needle sticks and possible contamination from small-diameter probes. The container of the inventions also provides a mechanism for restricting access to the probe to prevent its reuse, and a method for its safe attachment to and detachment from an ultrasonic medical device. The inventions restrict reuse of the medical device by retaining the used device in a container that is not readily accessed by a user; the user must choose to use the container in order for the medical device to be removed and contained. The inventions do not address a means to render the used device incapable of further use, nor do the inventions address potential reuse of the device, with the user not restricted from reusing the device, even if already used with a patient. There remains a need for a probe sheath system that prevents reuse of a sheath and that provides a means and method of its safe removal from the probe, without the need for handling of the sheath by the user.

### PROBLEM TO BE SOLVED

There remains a need for a probe sheath system that prevents reuse of a sheath or cap and that provides a means and method of its safe removal from the probe, without the need for handling of the sheath or cap by the user. In specific, there remains a need for a probe sheath/cap system that prevents reuse of a sheath through features of the sheath and probe system that cannot be readily contravened by a user, and that protect the user from handling biologically contaminated surfaces of the probe sheath system during removal of a used sheath.

### SUMMARY OF THE INVENTION

This invention relates to a medical device comprising at least one bioprobe for collecting data, a single-use protective cover mechanically attached to at least one bioprobe, a control module, and a sheath removal system. The sheath removal system comprises a receiving port for at least one bioprobe with an attached single-use protective cover, a single-use protective cover locking mechanism for retaining the single-use protective cover, and a single-use protective cover removal feedback mechanism in communication with the control module.

The invention also relates to a medical device comprising at least one bioprobe for collecting data, a single-use protective cover mechanically attached to the bioprobe, a control module, and a destructive sheath removal system comprising a receiving port for the bioprobe with an attached single-use protective cover, a single-use protective cover locking mechanism for retaining the single-use protective cover, a single-use protective cover removal feedback mechanism in communication with the control module, and a single-use protective cover destruction mechanism.

The invention further relates to a medical device wherein the sheath removal system causes the collected data from at least one bioprobe to be locked so that the user cannot access these data until the single-use protective cover has been captured and retained by the single-use protective cover locking mechanism of the sheath removal system.

The invention also relates to a medical device wherein the sheath removal system causes the collected data from at least one bioprobe to be locked by the control module so that the user cannot use the bioprobe with a new patient until the single-use protective cover has been captured and retained by the single-use protective cover locking mechanism of the sheath removal system.

The invention also relates to a medical device wherein the single-use protective cover is mechanically attached to at least one bioprobe by a bayonet attachment.

The invention further relates to a medical device wherein the mechanical attachment of the single-use protective cover to at least one bioprobe is adjustably positionable.

Additionally, the invention relates to a medical device wherein destruction comprises rendering the sheath incapable of transmitting some or all of the illumination light.

Further, the invention relates to a medical device comprising a disposal unit for a single-use protective cover after destruction by the sheath removal system.

Additionally, the invention relates to a medical device wherein the destruction is a mechanical destruction selected from the group consisting of crushing, bending, cutting, breaking, or otherwise physically deforming said single-use protective cover. The destruction mechanism may also include means to reduce the ability of light to be transmitted by the sheath, including the application of paint, ink, polymer coatings to the sheath, and preferably to the distal portion of the sheath.

The invention further relates to a method of use comprising supplying at least one bioprobe for collecting data; mechanically attaching a single-use protective cover said at least one bioprobe; placing said at least one bioprobe with said mechanically attached single-use protective cover proximal a biological tissue to be interrogated; removing said at least one bioprobe with said mechanically attached single-use protective cover from said biological tissue after collecting data; placing said at least one bioprobe with said mechanically attached single-use protective cover into a sheath removal system comprising a receiving port for said at least one bioprobe with said attached single-use protective cover, a single-use protective cover locking mechanism for retaining said single-use protective cover, a single-use protective cover removal feedback mechanism in communication with said control module, and a medical waste container for retaining said single-use protective cover upon its removal from said bioprobe; locking said at least one bioprobe with said mechanically attached single-use protective cover into said locking mechanism to trigger said single-use protective cover removal feedback mechanism in communication with a control module to release the collected data to the control module; removing said at least one bioprobe for collecting data from the sheath removal system; and retaining said single-use protective cover in said medical waste container.

In addition, the invention relates to a method comprising supplying at least one bioprobe for collecting data; mechanically attaching a single-use protective cover said at least one bioprobe; placing said at least one bioprobe with said mechanically attached single-use protective cover proximal a biological tissue to be interrogated; removing said at least one bioprobe with said mechanically attached single-use protective cover from said biological tissue after collecting data; placing said at least one bioprobe with said mechanically attached single-use protective cover into a sheath removal system comprising a receiving port for said at least one bioprobe with said attached single-use protective cover, a single-use protective cover locking mechanism for retaining said single-use protective cover, a single-use protective cover removal feedback mechanism in communication with said control module, a single-use protective cover destruction mechanism, and a medical waste container for retaining said single-use protective cover upon its removal from said bioprobe; locking said at least one bioprobe with said mechanically attached single-use protective cover into said locking mechanism to trigger said single-use protective cover removal feedback mechanism in communication with a control module to release the collected data to the control module; removing said at least one bioprobe for collecting data from the sheath removal system; destroying the single-use protective cover retained in the locking mechanism with the destruction mechanism; and retaining said single-use protective cover in said medical waste container.

### ADVANTAGEOUS EFFECT OF THE INVENTION

The present invention includes several advantages, not all of which are incorporated in a single embodiment. The present invention comprises a bioprobe system for human health and screening and diagnosis incorporating a sheath that provides a sterile interface between a bioprobe and the human subject. The bioprobe system provides a means to control the use of a probe sheath used with a medical device, allows its disposal after use, and protects the user from possible contamination from biohazards that may be retained on the surface of the sheath. In specific, the bioprobe system includes a sheath removal system that allows the user to remove the sheath from a bioprobe without handling the sheath directly after use with a human subject. The system and method of the present invention provide a mechanism to prevent sheath reuse, whereby data collected with the probe are not released to the user until the probe sheath is processed by the removal and disposal system.

### BRIEF DESCRIPTION OF THE DRAWINGS

FIG. 1 represents the BioProbe System with bioprobe, sheath, control module, illuminator module, and sheath removal system.

FIG. 2 represents the bioprobe with sheath attachment features.

FIG. 3 represents a second embodiment of the bioprobe with attached sheath and handle features for incorporation of one or more light sources. FIG. 3A illustrates one means for incorporation of the one or more light sources. FIG. 3B represents the bioprobe with attached sheath and enclosed one or more light sources, with an external electrical cord for powering the one or more light sources. FIG. 3C represents the probe with attached sheath and enclosed one or more light sources, with a power cord running to an external power source for powering the one or more light sources. FIG. 3D represents the bioprobe with attached sheath and enclosed one or more light sources, with a power cord running to an external battery for powering the one or more light sources.

FIG. 4 represents a schematic of one embodiment of a sheath including the mechanical features that mate with the probe attachment features.

FIG. 5 represents a schematic of a sheath comprising a three part assembly.

FIGS. 6A and 6B illustrate the sheath of FIGS. 4 and 5, depicting the attachment and release features.

FIG. 7 represents a schematic of the bioprobe mated with a sheath, illustrating one means of mating the sheath to the probe.

FIG. 8 represents one embodiment of a sheath removal system, incorporating the receiving port for the bioprobe with sheath, the sheath locking features, sheath removal feedback features, sheath crushing mechanism, and disposal bag.

FIGS. 9A and 9B represent an embodiment of the sheath removal system, integrated with other components of the BioProbe System, depicting a bioprobe with attached sheath, both prior to insertion into the sheath removal system, and when inserted into the sheath removal system.

FIG. 10 represents another embodiment of the sheath removal system.

FIGS. 11A-11E—represent one embodiment of the sheath removal system and its use.

FIG. 11A depicts the sheath removal system with sheath reception port prior to insertion of the bioprobe with sheath.

FIG. 11B depicts the sheath removal system with the bioprobe and sheath inserted into the sheath reception port, so to enable sensing of the bioprobe and sheath in the sheath removal system and to allow unlocking of the screening data.

FIG. 11C depicts actuation of bioprobe and sheath in the sheath reception port so to enable sheath removal.

FIG. 11D depicts removal of the bioprobe and retention of the sheath in the sheath removal system.

FIG. 11E depicts deformation of the sheath in the sheath removal system and storage of the deformed sheath in the hazardous waste container.

FIG. 12 represents a method of use of a bioprobe with sheath and sheath removal system.

FIGS. 13A, 13B, 13C, and 13D represent another embodiment of the sheath removal system and its use.

FIG. 14 represents another method of use of a bioprobe with sheath and sheath removal system.

### DETAILED DESCRIPTION OF THE INVENTION

In the following detailed description, for purposes of explanation and not limitation, exemplary embodiments disclosing specific details are set forth in order to provide a thorough understanding of the claimed invention. However, it will be apparent to one having ordinary skill in the art having had the benefit of the present disclosure, that the claimed invention may be practiced in other embodiments that depart from the specific details disclosed herein. Moreover, descriptions of well-known devices, methods and materials may be omitted so as to not obscure the description of the claimed invention.

The present invention relates to a method and apparatus for human health screening, diagnosis, and imaging, using a non-invasive light-based technique that is conducive to safety and ease of use in both medical facilities as well as in remote care locations.

“Sheath”, “cap”, “cover”, or “protective cover” as used herein, refers to a device for covering or encasing, in whole or in part, a bioprobe or portion thereof connected to a light-based system for screening and/or diagnosing health and medical conditions of a patient. Throughout this description, “BioProbe” refers to a medical device that is used to screen for, diagnose, or monitor health conditions of human or animal subjects through interaction of the bioprobe with the animal or human tissue under test. More specifically, the bioprobe may comprise a light-based device that is used to screen, diagnose, or monitor health and medical conditions of a patient, by illuminating a skin or tissue of the patient with light, and measuring light that is returned to the bioprobe. “Container” as used herein refers to an apparatus used for safe removal, storage, and disposal of a medical article or device, in most particularly, the sheaths used with a bioprobe to provide a sterile interface between the bioprobe and the patient. Throughout this description, the terms “distal” and “proximal” when pertaining to description of a BioProbe and sheath are relative to the user, i.e. distal is away from the user and indicates the forward portion of the device, whereas proximal is nearest to the user and relates to the back portion of the device.

Further, throughout this description, the term “wireless” refers to a means of telecommunications wherein the transfer of information occurs between two or more points that are physically not connected. Distances can be short, as a few meters as in remote control; or long ranging up to thousands of kilometers for the communication of data for review, storage, and other applications. It encompasses various types of fixed, mobile, and portable two-way radios, cellular telephones, personal digital assistants (PDAs), and wireless networking. Other examples of wireless technology include GPS units, wireless computer mice, keyboards and Headset (telephone/computer), headphones, radio receivers, and satellite communications.

Destruction of the single-use protective cover refers to any mechanism that can be used to render the protective cover or the bioprobe used with the protective cover inoperable. For example, destruction of the protective cover may include crushing, cutting, bending or other means to deform the sheath. Other means of destruction can include, for example, coating the protective cover with an ink, paint, a dye, plastic, or other material that will adhere to the optical window portion and render the protective cover incapable of transmitting light as required by the BioProbe System.

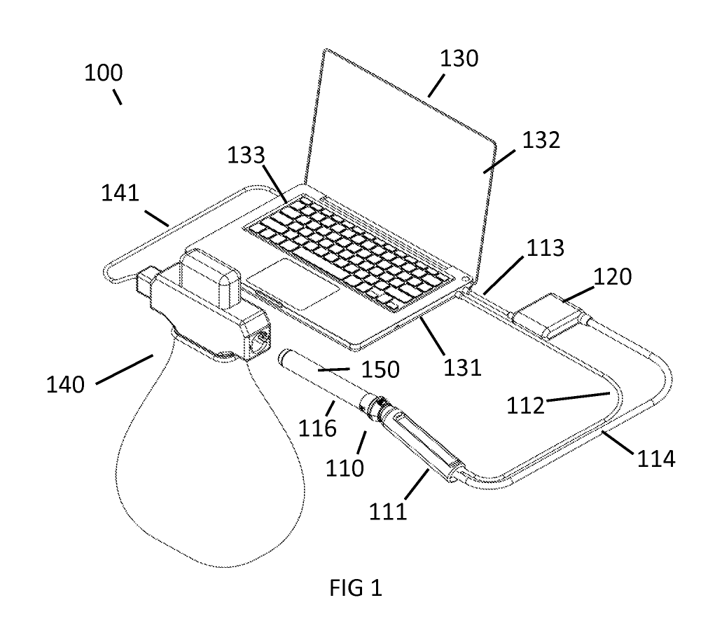

FIG. 1 represents a BioProbe System 100 with bioprobe 110, illuminator module 120, control module 130, sheath 150, and sheath removal system 140. The BioProbe System 100 includes one or more measurement bioprobes; one bioprobe 110 is illustrated, but additional bioprobes may be utilized so to measure different tissues of the body, and to enable measurement of tissues that are located in differently sized or shaped human orifices. The BioProbe System envisioned herein includes the disclosures of US 2012/0232408, having a Sep. 13, 2012 filing date, with the entirety of that patent application incorporated herein by reference.

The bioprobe 110 includes one or more optical detectors to measure the emitted light, such as fluorescence and reflected light. The one or more detectors may be selected from diodes, CCDs, CMOS, with one or more photosensitive regions to sense the strength of emitted fluorescence. The one or more detectors can use one or more optical filters to select the specific wavelengths to be sensed. The optical filters may be cut-off filters, narrow-band filters, band edge filters, and can include beam splitters, plate filters, flexible filters, gratings, and other spectrally selective components known to those skilled in the art.

A bioprobe body 116 serves to enclose the components that illuminate the tissue and that receive the fluorescence, reflected light, and scattered light emitted by the tissue. Depending on the embodiment, the bioprobe body 116 may comprise plastic, glass, or metal materials, with transmissive windows and protective sheaths. The bioprobe body may incorporate one or more switching features (not shown) to initiate a measurement and to control transmission of the illuminating light by the bioprobe. The proximal portion of the bioprobe body 116 includes a bioprobe handle 111.

The illuminator module 120 includes one or more light sources used to illuminate the tissue under examination. The one or more light sources may be one or more of an arc lamp with a narrowband filter, an LED with or without an optical filter, a fiber-coupled source, a laser, or another luminous emitter that has sufficient light energy in the desired optical band to excite fluorescence at the tissue site. The one or more light sources may be located within the bioprobe 110; they also may be located in a separate illuminator module 120 as indicated in FIG. 1. The illuminator module 120 is coupled by one or more optical waveguides 114 to the bioprobe 110. The light that is conducted from the illuminator module 120 to the bioprobe 110 is conducted through the interior of the bioprobe 110 to the distal end of the bioprobe where it exits the bioprobe through an optical window. The illuminator module 120, while shown as a distinct element of the BioProbe System 100, may be attachable to or incorporated within the bioprobe itself.

The BioProbe System 100 further includes a control module 130 that comprises a processor 131, display 132, keyboard 133, wired or wireless connections 112 to the bioprobe 110, wired or wireless connections 113 to the illumination module, the illuminator module 120, and a power and data cord 141 to the sheath removal system 140. The processor 131 is used to power portions of the BioProbe System, to run the measurement, to collect the measured data, to analyze the measured data so to render screening information concerning the health status of the tissue under test, to display the captured images, to display the modified images with identification of normal and abnormal tissues, and to store the measured and processed data in addition to other information as required. The processor 131 may be a computer, a laptop, a microprocessor, an application specific integrated circuit (ASIC), analog circuitry, digital circuitry, or any plurality and/or combination thereof configured to implement the disclosed methods and their equivalents. The processor 131 may include or be configured to execute instructions stored on a computer readable storage medium for implementing the disclosed methods and their equivalents. Depending on the embodiments, configurations may include instructions to control variables such as exposure duration, illumination strength, total exposure, data collection and organization, data analysis, data review and display.

Additionally or optionally, the BioProbe System 100 may include a data communicator wherein data collected by the bioprobe are transmitted to the processor and/or to another data receiver (for example, to a database). Such a data communicator may include, but is not limited to, a radio frequency (RF) transmitter and/or receiver. In other embodiments, data may be transmitted by satellite to enable remote data storage, analysis, retrieval, and other features.

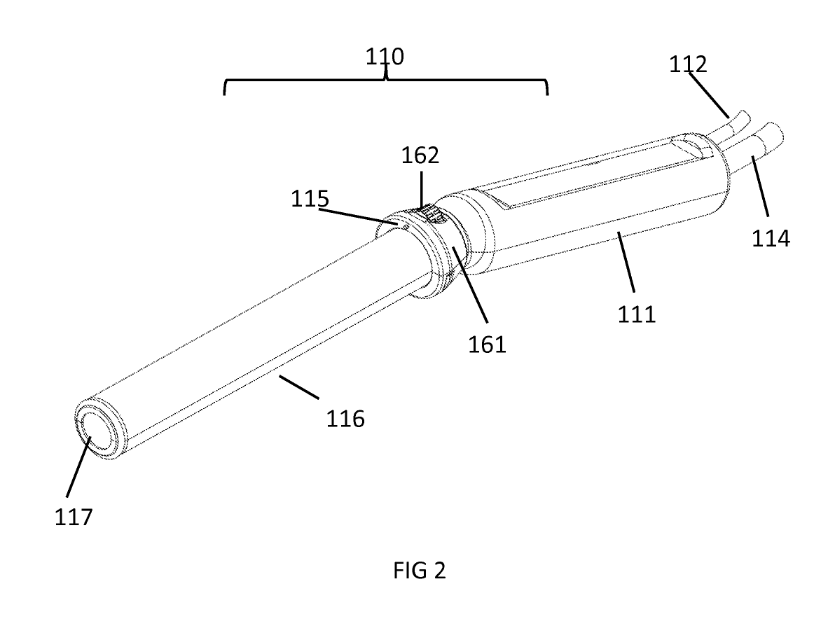

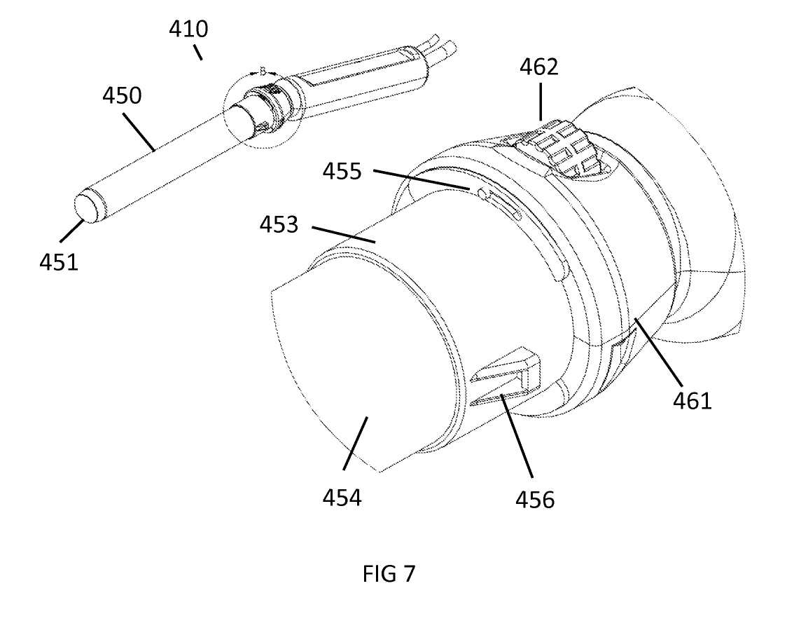

FIG. 2 represents the bioprobe 110 with sheath attachment features 115. The bioprobe 110 includes a handle 111 at the proximal end of the bioprobe, a largely cylindrical body 116, and a distal optical window 117. The bioprobe 110 may also include a wired connection 112 to the control module and one or more optical waveguide connections 114 to one or more illuminators. The bioprobe incorporates sheath attachment features 115 providing a means for attachment of a protective sheath (not shown). One embodiment of the sheath attachment features is illustrated in FIG. 2, comprising a bayonet mount. Other attachment features may include screw threads, latching features, and other equivalent connection means. Bayonet mounting features provide an advantage of being readily facile to mold using plastic materials, using standard mold tooling. The protective sheath provides a sterile interface between the bioprobe and the patient. The bioprobe includes a focus wheel 162 and focus carriage 161. These elements allow the sheath position to be adjusted along the length of the bioprobe body 116. This is explained in further detail with the description of FIG. 7, below.

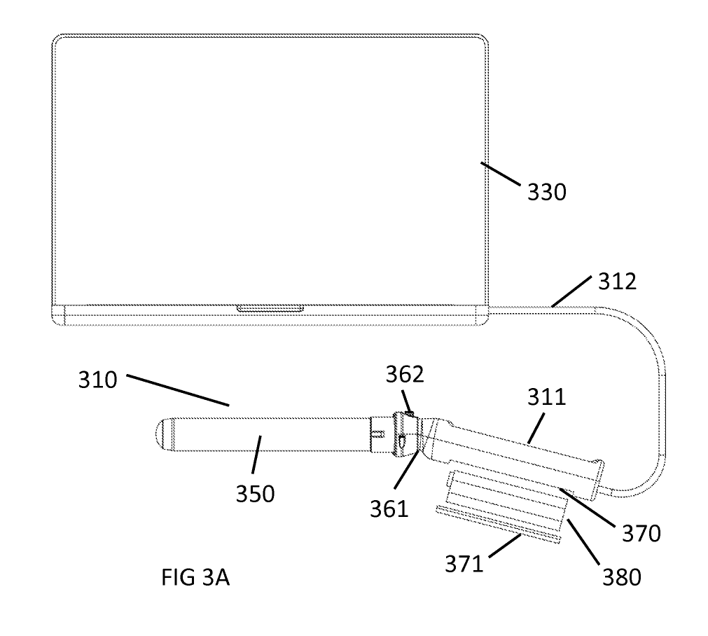

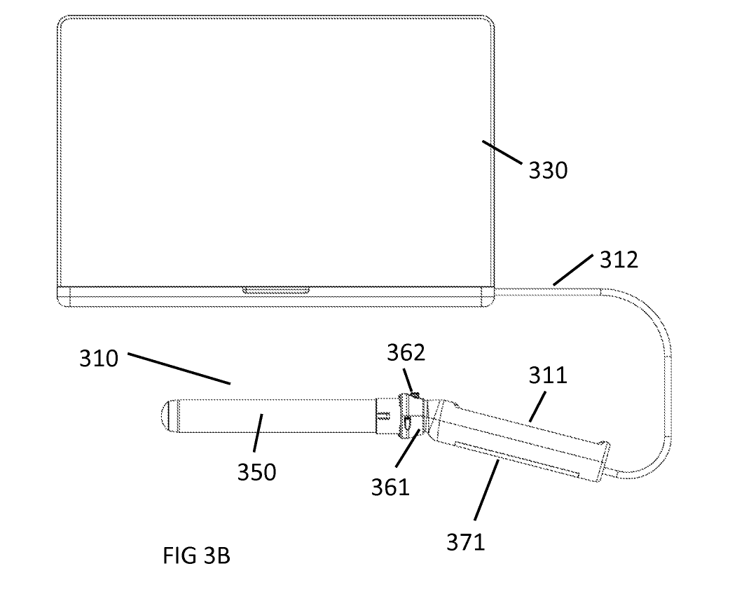

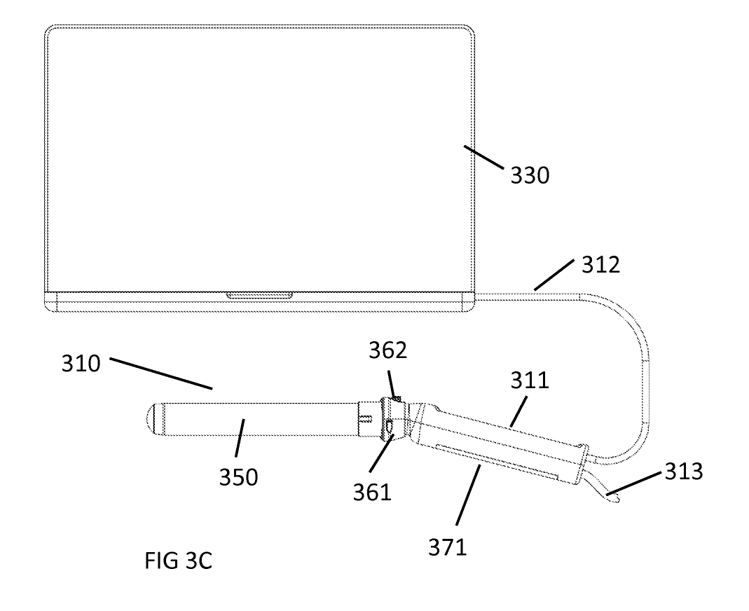

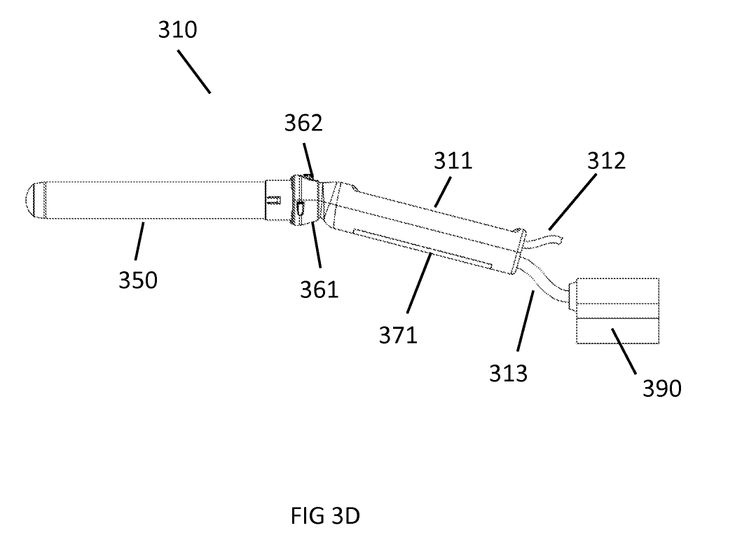

FIGS. 3 represents a second embodiment of the bioprobe 310 with an attached sheath 350 and handle features including recess 370 and attachment cover 371 for incorporation of one or more light source modules 380. Bioprobe 310 is illustrated with a connection 312 to the control module 330, with the connection exiting the bioprobe through the proximal portion of the handle 311. This connection allows for powering of the bioprobe 310 as well as data transfer between the bioprobe and the control module. Data transfer may alternatively be conducted via wireless communications. Also shown in FIGS. 3 are focus wheel 362 and focus carriage 361. These elements allow the position of sheath 350 to be adjusted along the length of the bioprobe body. FIG. 3A illustrates one means for incorporation of the one or more light sources. In this embodiment, the BioProbe 310 includes the bioprobe handle 311, into which is built recess 370 for receiving the one or more light source modules 380. The light source modules 380 comprise the one or more LEDs, diodes, lamps, laser sources or other lighting elements known to those skilled in the art. In addition, the light source modules may include one or more batteries to power the one or more light sources (not shown). The light source modules 380 further includes a receptacle for the one or more light sources and batteries (if included), as well as features that permit the light source module to mate to and be optically aligned with the other elements internal to the bioprobe. The one or more light sources may be powered by batteries that are built into the light source modules 380. FIG. 3B illustrates the bioprobe of FIG. 3A with the attachment cover 371 comprising a panel that seals the handle 311 after incorporation of the one of more light source modules 380.

If batteries are not included integral to the light source modules 380, external power may be supplied. This power option is illustrated in FIG. 3C which represents the bioprobe 310 with attached sheath 350, enclosed one or more light source modules (not shown), and external power cord 313. This external power cord 313 may replace the fiber cable 114 shown in FIG. 1 and FIG. 2, where the fiber cable 114 otherwise connects the bioprobe 110 to an external light source. In FIG. 3C, the light source module (not shown) is integrated into the bioprobe handle 311, with attachment cover 371 sealing the light source module into the recess 370 (not shown) in the handle. Power cord 313 may lead to various external power sources including standard AC power, DC power with power transformers as required, and/or external battery power. The option of using an external battery to power the one or more light source modules is represented in FIG. 3D.

FIG. 3D represents the bioprobe 310 with attached sheath 350 and enclosed one or more light sources (not shown), with an external power cord 313 running to an external battery pack 390 for powering the one or more light source modules 380 (not shown). For ease of use, the power cord 313 connecting to the battery pack 390 may include a connector that facilitates rapid and easy connection to and from the batter pack 390. In addition, the power cord 313 and/or battery pack 390 may include a power switch so that batter power may be enabled or disabled to the one or more light source modules 380 (not shown).

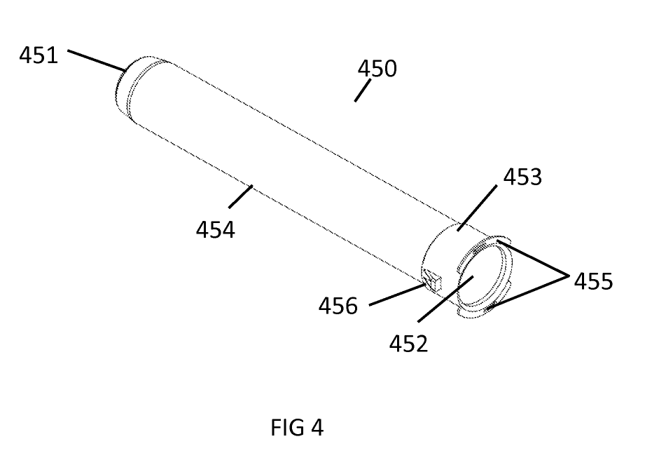

FIG. 4 represents a schematic of one embodiment of a sheath 450 including the mechanical features 455 that mate with the bioprobe attachment features 115 (see FIG. 2). The sheath 450 comprises a largely cylindrical sheath body 454 with an opening 452 in the proximal end near the sheath base 453, and an optically transparent window 451 in the distal end. The proximal end comprises one or more mechanical features 455 that engage the sheath with one or more features that are incorporated into the bioprobe body. The mating features permit an attachment of the sheath to the bioprobe. One embodiment of the sheath attachment features is illustrated in FIG. 4, comprising a bayonet mount. Other attachment features may include screw threads, latching features, and other equivalent connection means. Bayonet mounting features provide an advantage of being readily facile to mold using plastic materials, using standard mold tooling. The mating features 455 may also enable a mechanical adjustment of the sheath relative to the bioprobe body, so to adjust the focus of the bioprobe. This mechanical adjustment shifts the longitudinal position of the distal window surface of the sheath relative to the distal window of the bioprobe body. The sheath further includes mechanical features 456 that engage with mating features in the sheath removal system 140 (see FIG. 1). These mechanical features 456, when locked into the mating features of the sheath removal system 140, retain the sheath in the removal system and do not allow its removal from that system (see FIG. 1).

FIG. 5 represents a schematic of a second embodiment of a sheath comprising a three part assembly. The three part assembly is shown distinctly in FIG. 5 comprising the sheath window 551, sheath body 554, and sheath base 553. The sheath window 551 is fabricated from materials that allow efficient light transmission in the 290 nm-1000 nm optical spectrum and preferably in the 290 nm-700 nm optical spectrum. Preferably, the sheath window 551 is fabricated from a material that permits light transmission greater than 85%, more preferably greater than 90%. The sheath window 551 depicted in FIG. 5 includes a curved optically transmitting surface 551 and a cylindrical portion 559. The cylindrical portion 559 is fabricated to connect to the cylindrical sheath body portion 554 at the distal end of the sheath body portion 554. This sheath body portion 554 is fabricated of a rigid plastic material, preferably formed by extrusion. While other forming methods may be used, extrusion of the sheath body portion 554 is potentially a low cost fabrication method to make this cylindrical part. The sheath body portion 554 should be formed from a sufficiently stiff and thin-walled tubular plastic so to facilitate crushing, cutting, bending or other means to deform the sheath after use. A sheath base 553 is the third part of the sheath assembly, and includes one or more mechanical features 555 that engage the sheath 550 with one or more features that are incorporated into the bioprobe body. The sheath further includes mechanical features 556 that engage with mating features in the sheath removal system 140 (see FIG. 1). The sheath base 553 is preferably molded of a plastic material that allows sufficient strength in the mechanical features 556 so that they are readily retained by the sheath removal system 140 of FIG. 1. Sheath base 553 may be fabricated of the same plastic material as sheath body 554, or may be fabricated from other plastics that may be readily joined to sheath body 554. Joining methods used to attach the sheath window 551 and sheath base 553 to the sheath body 554 include laser welding and adhesive attachment. Thermal expansion may also be used to attach one or both of the sheath window 551 and sheath base 553 to the sheath body 554. Differential thermal expansion allows heated or cooled parts to be assembled by inserting the parts, one into another. The overlapping areas of the parts become attached due to differential thermal expansion when the heated or cooled parts reach ambient or operating temperature.

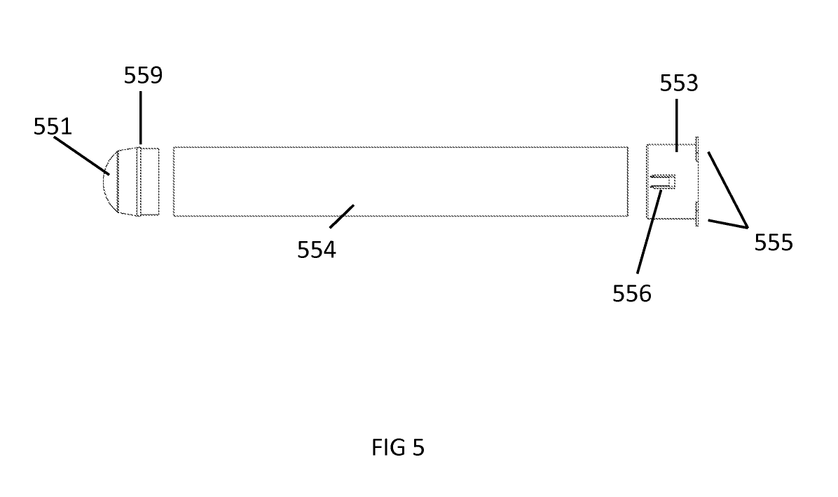

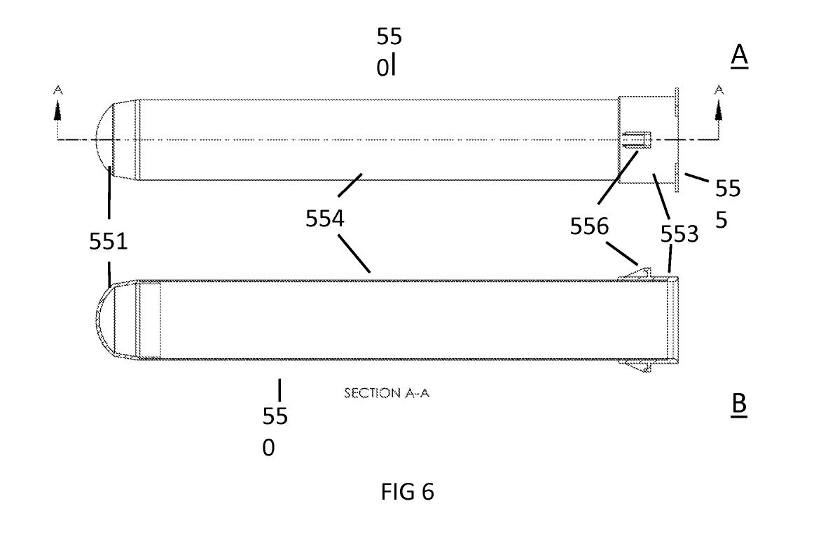

FIGS. 6A and 6B illustrate the sheath of FIGS. 4 and 5, depicting the attachment and release features FIG. 6A depicts one view of the sheath 550 comprising the sheath window 551, sheath body 554, and sheath base 553. Also shown are the sheath attachment features 555, comprising mechanical features 555 that engage the sheath with one or more features that are incorporated into the bioprobe body. The mating features permit an attachment of the sheath to the bioprobe body. A second view of sheath 550 is depicted in FIG. 6B, illustrating the release features 556. These mechanical release features 556, when locked into the mating features of the sheath removal system 140 (see FIG. 1, for example), retain the sheath in the removal system and do not allow its removal from that system.

FIG. 7 represents a schematic of the sheath 450 that is mated with the bioprobe 410. As a non-limiting example, this figure illustrates the sheath 450 of FIG. 4. The sheath 450 comprises a largely cylindrical sheath body 454 with an optically transparent sheath window 451 in the distal end. The proximal end of the sheath 450 comprises one or more mechanical features 455 that engage the sheath 450 with one or more features that are incorporated into the bioprobe body. The mating features permit an attachment of the sheath to the bioprobe; this attachment of the sheath 450 to the bioprobe also allows a mechanical adjustment of the sheath relative to the bioprobe body, so to adjust the focus of the bioprobe. This mechanical adjustment shifts the longitudinal position of the distal window surface of the sheath relative to the distal window of the bioprobe body. As shown in FIG. 7, the mechanical features in one embodiment comprise a focus carriage 461 and a focus wheel 462. Rotation of the focus wheel causes the sheath position to shift longitudinally. The window 451 of the sheath 450 permits transmission of the one or more illumination light sources to the tissue under examination. Sheath window 451 further defines an optical surface close to the object plane of the optical system of the bioprobe; the object plane comprises the surface of the biological tissue under examination. As the position of sheath window is adjusted longitudinally relative to the optical system contained within the bioprobe body, the focus of the object plane is adjusted, allowing the image quality of the biological tissue under examination to be adjusted. The focus of the optical imaging systems may be controlled through adjustment of the focus wheel 462 that controls the sheath position via mechanical features in the sheath and the bioprobe body. The window of the sheath 451 further permits transmission of the light reflected from the tissue under examination, as well as transmission of fluorescence emission from the tissue. The materials comprising the window of the sheath 451 are selected to permit the required optical performance. The sheath 450 further comprises mechanical release features 456 that are used to retain the sheath in the sheath removal system after use of the bioprobe with a single patient. The mechanical release features 456 are fabricated as part of the sheath base 453. All materials used in the manufacture of the sheath are selected to meet rigorous requirements for insertion into the human body, or for contact with tissues of the human body. Further, the materials are selected to be able to be sterilized.

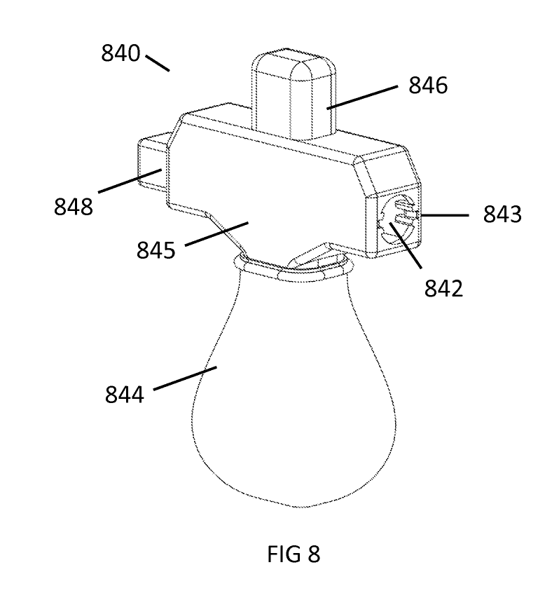

FIG. 8 represents the sheath removal system 840 as a stand-alone module, having a sheath removal body 845 incorporating the receiving port 842 for the bioprobe with sheath, the sheath locking features 843, sheath removal feedback module 848, and the sheath crushing mechanism 846. A disposable medical waste bag 844 is attached to the sheath removal body 845.

This sheath removal system 840 provides a means to remove the sheath from the bioprobe body following use of the bioprobe system with a patient. A used sheath may have bodily fluids retained on its surface; if used in patients with active infections or diseases such as HIV, it is preferred that the sheath be removed without contact by the health care worker providing the screening test. The sheath removal system 840 comprises a receiving port 842 into which the distal end of bioprobe with attached sheath is inserted. As the bioprobe with attached sheath is inserted, the mechanical features 456 of the sheath (see FIG. 4) engage with mating features 843 located within the receiving port 842 of the sheath removal system 840. These mechanical features 456, when locked into the mating features 843, retain the sheath in the removal system 840 and do not allow removal of the sheath from that system. One embodiment of the mating features 843 is illustrated in FIG. 8 and comprises angled elements with sufficient spring force to allow the mechanical features 456 of the sheath to slide over mating features 843, with these mating features 843 then snapping behind the mechanical features 456 to lock the sheath in place.

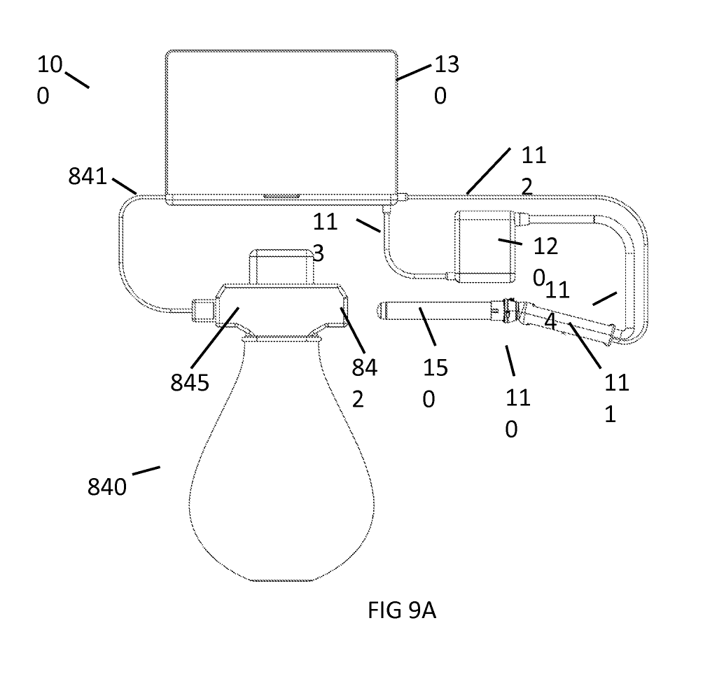

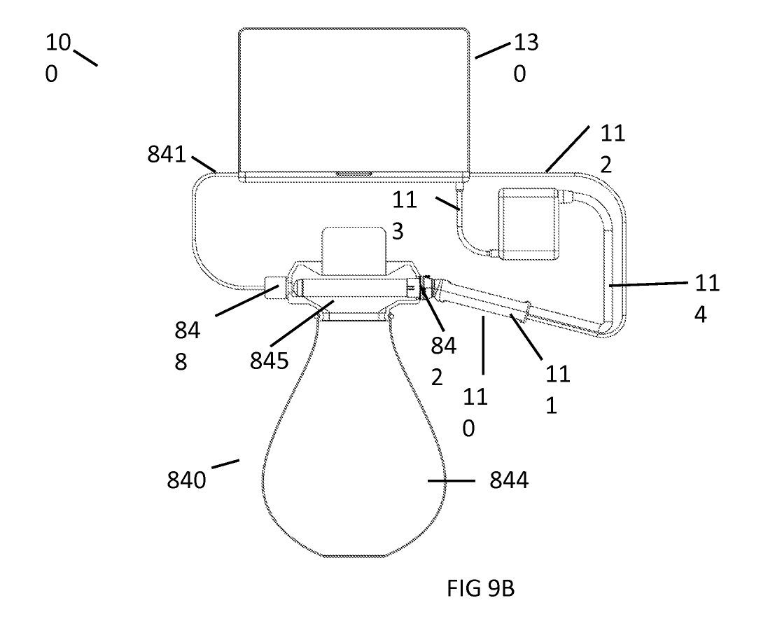

FIGS. 9A and 9B represent an embodiment of the sheath removal system integrated with other components of the BioProbe System 100. Both FIGS. 9A and 9B illustrate an embodiment of the BioProbe System 100 including the control module 130, bioprobe 110 and sheath removal system 840. The BioProbe System of both figures includes illuminator module 120, coupled by one or more optical waveguides 114 to the bioprobe 110 having bioprobe handle 111. The illuminator module 120, while shown as a distinct element of the BioProbe System 100, may be attachable to or incorporated within the bioprobe 110 itself.

The BioProbe System 100 of FIGS. 9 further includes wired or wireless connections 112 between the control module 130 and the bioprobe 110, wired or wireless connections 113 to the illumination module, and a power and data cord 841 connected to the sheath removal system 840. FIG. 9A depicts a bioprobe 110 with sheath 150 prior to insertion into the sheath removal system 840. The bioprobe is shown oriented so that the distal end of the sheath is directed toward receiving port 842 that is a part of the sheath removal body 845. FIG. 9B depicts a bioprobe 110 with sheath inserted into the sheath receiving port 842 of the sheath removal system 840. When inserted into the sheath receiving port 842, the distal portion of the bioprobe is positioned in front of sheath removal feedback module 848. This module includes one or more optical detectors (not shown) and a power and data cord 841 that is connected to control module 130. The detector is used to confirm the presence of the bioprobe with sheath in the sheath removal system, prior to removal of the sheath.

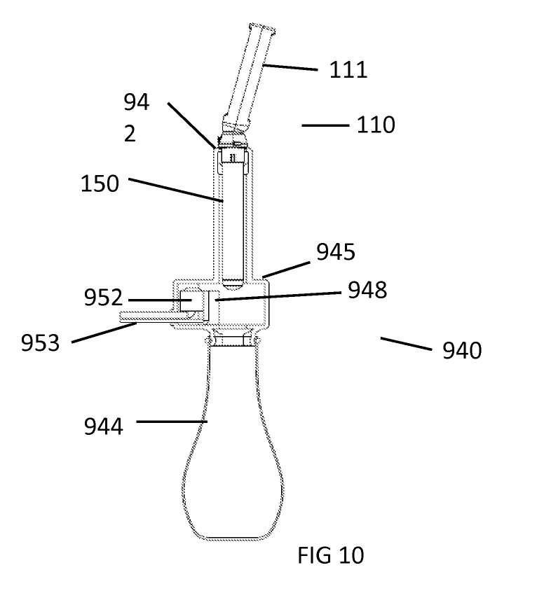

A second embodiment of a sheath removal system is shown schematically in FIG. 10. The sheath removal system 940 comprises a sheath removal body 945 incorporating the receiving port 942 to receive the bioprobe with sheath, the sheath locking features (not shown), sheath removal feedback module 948, and disposable medical waste bag 944 attached to the sheath removal body 945. The bioprobe 110 having handle 111 and attached sheath 150, is inserted into the receiving port 942 where the sheath engages with the sheath locking features (not shown) of the sheath removal system 940. Onto slide 953 are attached feedback module 948 and optional deposition module 952. Feedback module 948 includes one or more optical detectors (not shown) that are used to confirm the presence of the bioprobe with sheath in the sheath removal system. The optional deposition module 952 comprises an assembly that applies a thin coating to the distal end portion of sheath 150. The distal end portion may be coated using a spray applicator, roller, pad, or other equivalent means to deposit a partially or completely opaque coating. The partially or completely opaque coating may be an ink, paint, a dye, plastic, or other material that will adhere to the optical window portion and render the sheath incapable of transmitting light as required by the BioProbe System. The sheath removal system 940 may be used without the deposition module 952; in this case, the sheath is released into the medical waste bag 944 without being deformed or otherwise mechanically or optically altered. The operation of this embodiment of the sheath removal system is included in the examples that follow.

The bioprobe and sheath described above may comprise a bioprobe that is an endoscope, a bioprobe that is an imaging device, a bioprobe that uses ultrasound to form images, a bioprobe that uses light to form images, a bioprobe that collects light-based data from biological tissues, and similar devices used for human and animal health status screening and diagnosis. The invention described herein relates broadly to health screening, diagnostic, and monitoring equipment that includes the use of a sheath or other protective covering to form a barrier between the screening instrument and human or animal tissue under test. More specifically, the invention described herein relates to health screening, diagnostic, and monitoring equipment that includes a single-use sheath that covers at least a portion of the bioprobe, wherein removal and disposal of the sheath is required after use with a single subject. The health screening, diagnostic, and monitoring equipment described herein includes a sheath removal system that permits the protective, single-use sheath to be removed without the need for the user to handle the sheath directly. In addition, the sheath removal system causes diagnostic and screening data to be locked by the measurement system so that the user cannot access these data until the sheath has been captured and retained by the sheath removal system. The limited access to measured data may be accomplished in at least two manners.

In a first manner of limited access to measured data, images and measured light signals may be viewed by a health care provider during use of the bioprobe system, with the data not saved unless the sheath has been captured and retained by the sheath removal system. The bioprobe system may not be used on a new patient until the sheath is captured and retained by the sheath removal system, with the health care provider unable to start the system with a new patient until the sheath removal has been successfully implemented, and a new sheath attached to the bioprobe.

In a second manner of limited access to measured data, images and measured light signals may not be fully accessible to the health care provider until the sheath has been captured and retained by the sheath removal system. In the examples herein, the full breadth of options to limit access to measured data are intended, with the examples not intended to limit the scope of manners in which the data may be protected by the control module until the sheath has been removed and a new sheath applied to the bioprobe.

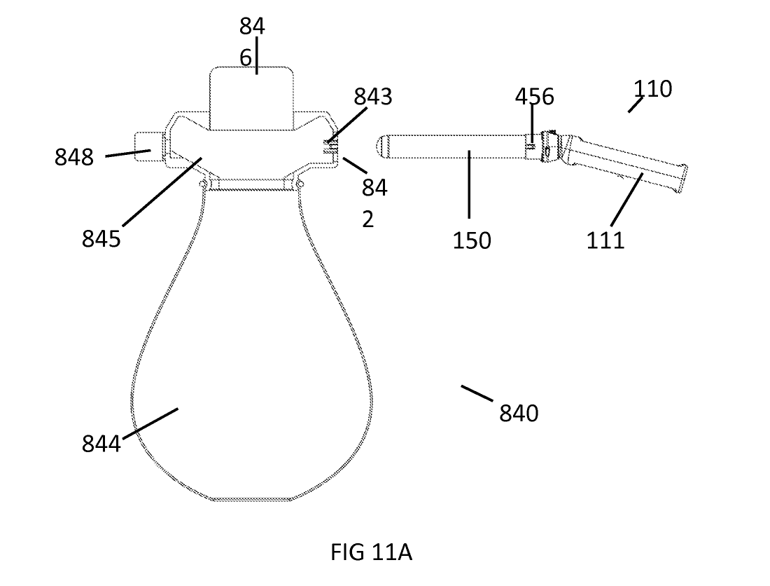

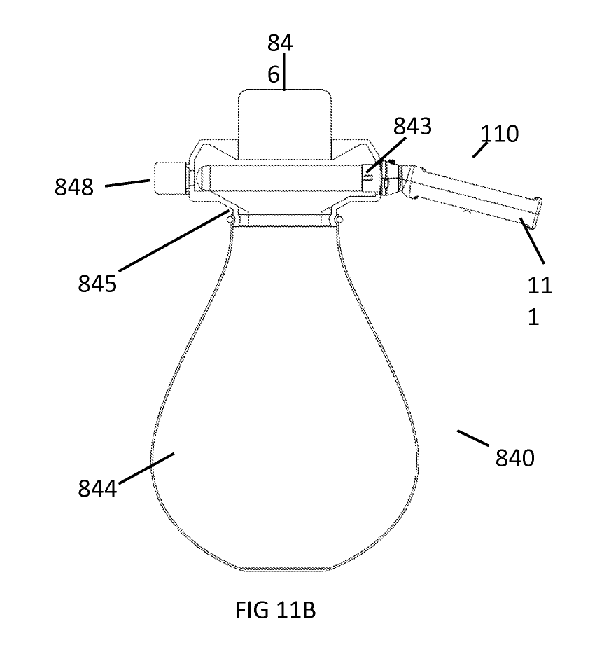

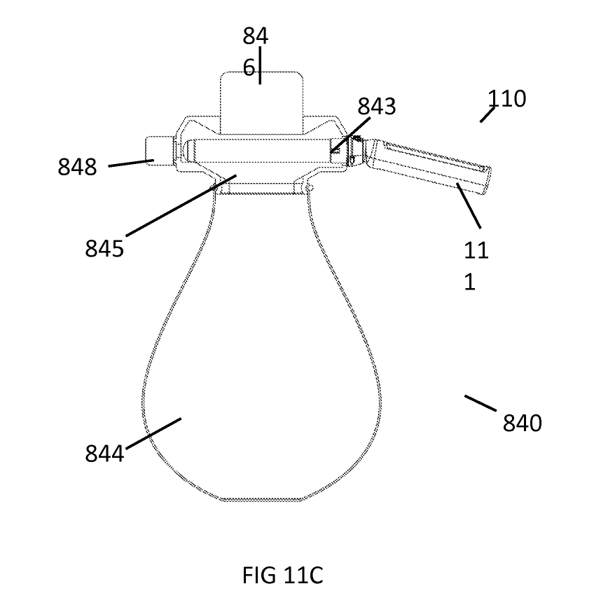

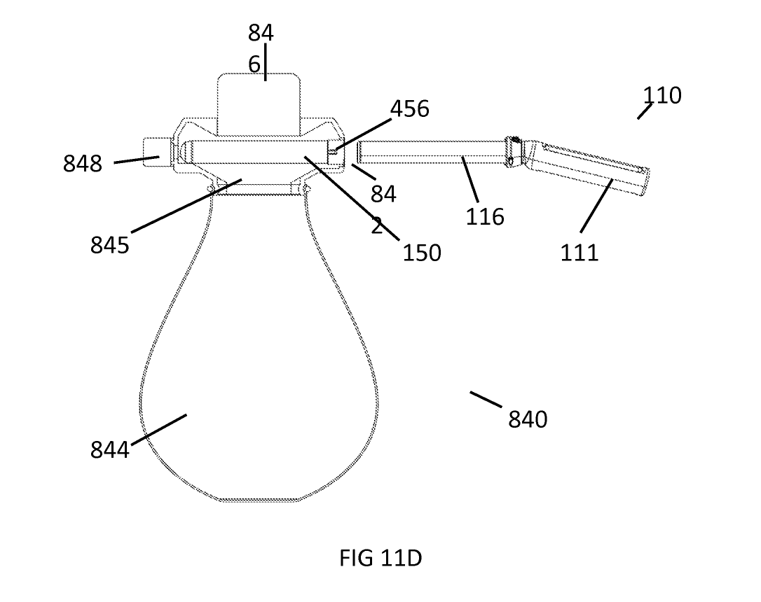

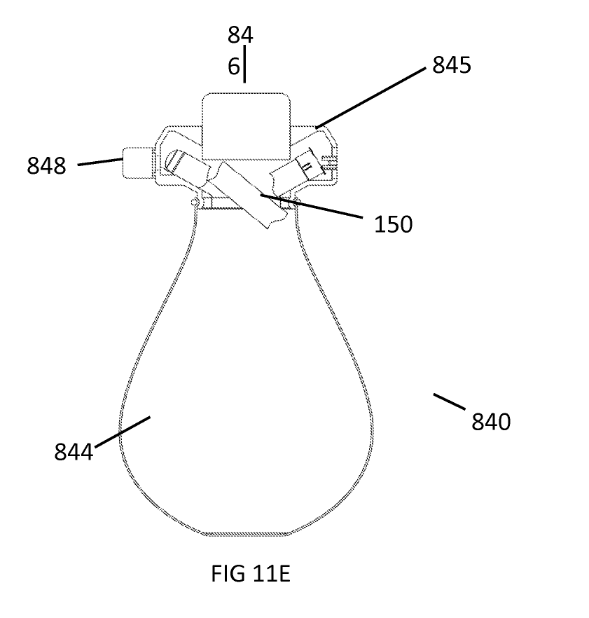

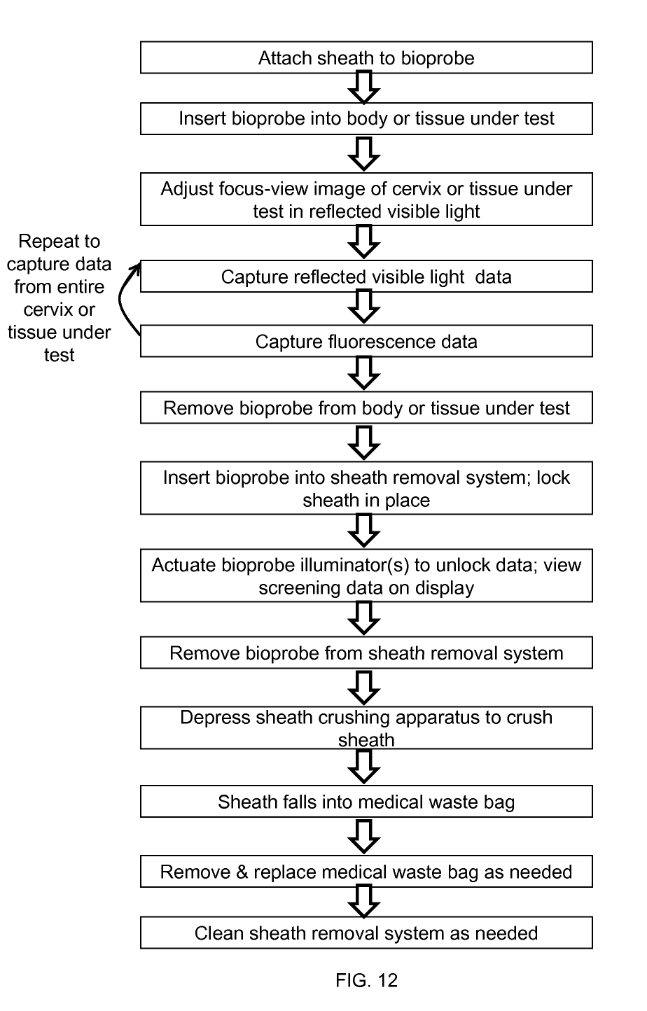

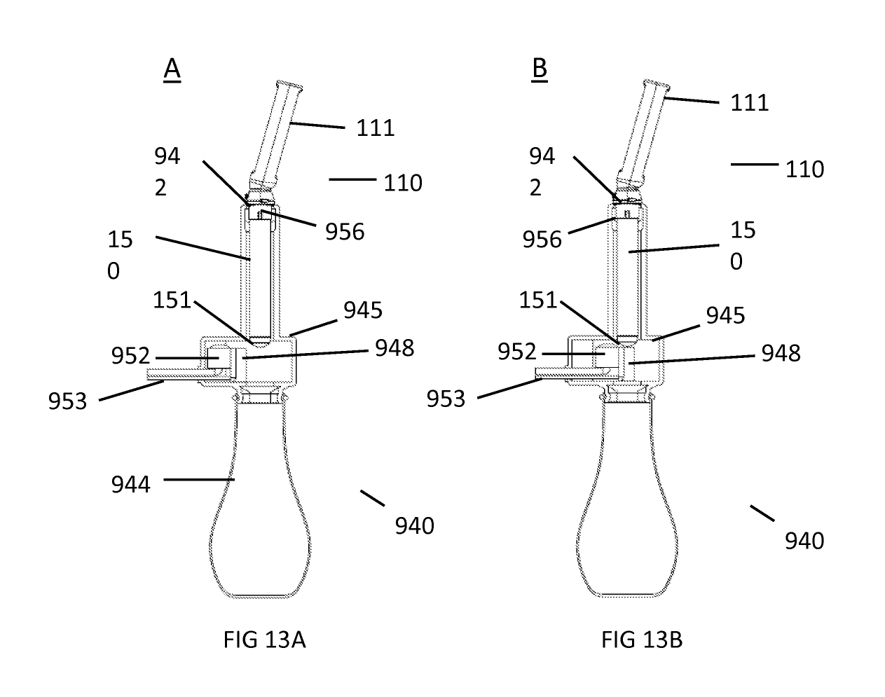

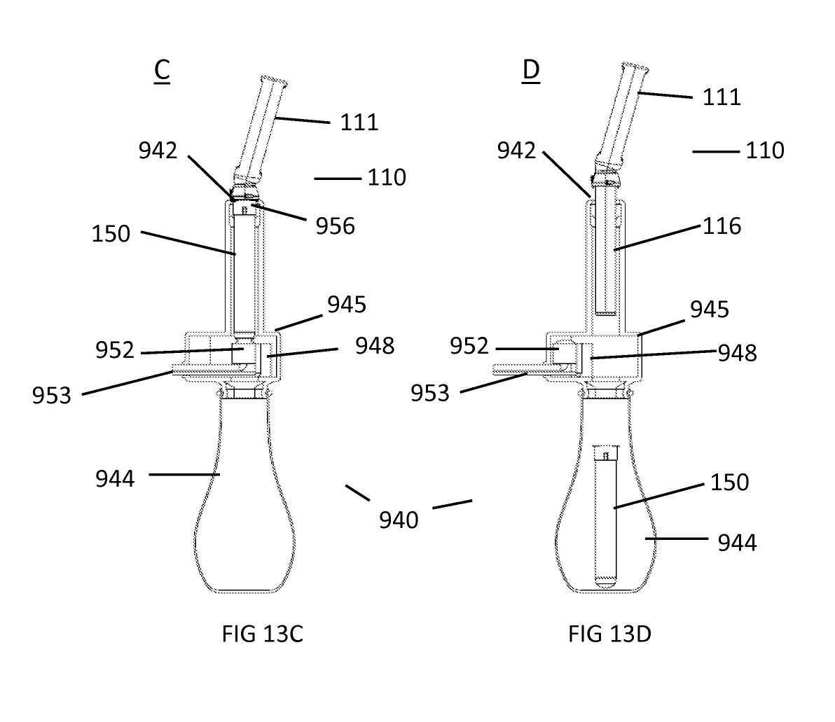

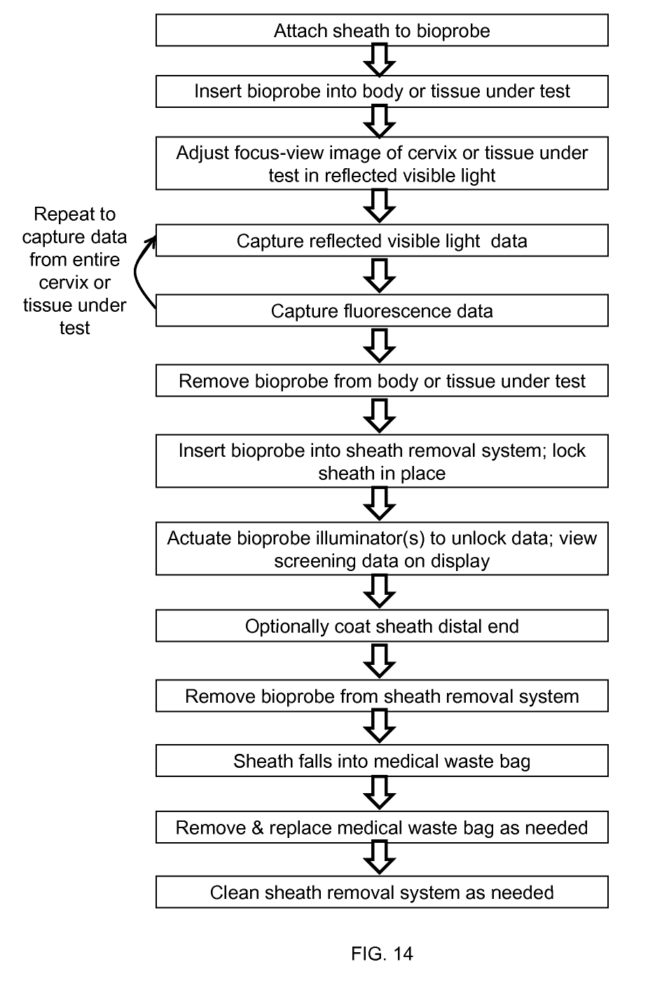

Two examples of the health screening equipment with sheath removal system are depicted in FIG. 11 and FIG. 13, with methods of use depicted schematically in the flow charts of FIG. 12 and FIG. 14. In specific, the health screening equipment of these examples comprises a bioprobe, but could equally well be a different screening system using a light-based method to interrogate a biological sample under test. These examples are not meant to be limiting, and are presented to be illustrative.

FIGS. 11A-11E represent the sheath removal system depicted earlier in FIGS. 8 and 9. FIG. 11A depicts a schematic of the sheath removal system 840 with reception port 842 prior to insertion of the bioprobe 110 with attached sheath 150. It is assumed that the bioprobe with sheath has been used to collect images and/or related data from a single patient. As the images and/or related data are collected, these are depicted on the display 132 of the control module 130 (see FIG. 1). While these images and/or related data may be viewed, the bioprobe system does not yet display screening information. Screening information includes identification of regions of human tissue where suspected abnormalities are located, the size of the suspected abnormal regions, the orientation of the abnormal regions with respect to surrounding tissues, the suspected degree of abnormality, and related information. Screening information is locked, and not made available to the person conducting the screening test, until the sheath removal system 840 acknowledges insertion and locking of the bioprobe 110 with sheath 150 into the bioprobe removal system 840. The bioprobe 110 with sheath 150 is inserted into the sheath removal system 840 by positioning the distal end of the bioprobe with optical window 151 in the reception port 842. The bioprobe is pushed into the reception port until the sheath release features 456 engage with the mating features 843 of the sheath removal system 840. Engagement of release features 456 and mating features 843 occurs when the bioprobe sheath 150 cannot be removed from the reception port 842.

FIG. 11B depicts a schematic of the sheath removal system reception port 842 with bioprobe 110 and attached sheath 150 engaged in the reception port 842. Prior to detachment of the sheath 150, one or more of the bioprobe illuminators (not shown) are pulsed on and off. The light emitted by the one or more illuminators is transmitted by the optical window 151 of the sheath 150, and impinges the feedback module 848 of the sheath removal system 840. The feedback module 848 includes one or more optical detectors and, optionally, one or more narrowband optical filters. The one or more narrowband optical filters are selected to transmit the light from the one or more bioprobe illuminators. When the pulses of light from the one or more illuminators are sensed, the bioprobe system control module acknowledges detection of the pulses of light; if acknowledgement is not done, the pulses of light may be transmitted again, until the control module acknowledges receipt of these signals. Pulsing of the one or more illumination signals in a predetermined manner allows the control module to differentiate these signals from ambient light. When these pulsed signals are acknowledged, the control module releases the screening data so that it may be viewed on the display 132 of the control module 130. Locking of the data by the control module, with release only when the sheath 150 is fully engaged in the reception port 842, insures that the sheath is used only for a single patient. Full engagement of the sheath in the reception port 842 may be more rigorously determined through addition of an interlock that senses engagement of the sheath release features 456 with the mating features 843 of the sheath removal system 840. Also illustrated in FIG. 11B is a disposable medical waste bag 844, attached to the sheath removal body 845.

FIG. 11C depicts a schematic of the bioprobe 110 with sheath 150 in the reception port 842, where the removal of the sheath 150 from the bioprobe 110 has been initiated. Removal of the sheath may be done by a rotation of the bioprobe 110, through rotation of the bioprobe handle 111 relative to the reception port 842. This rotation is done to detach the features that join the sheath 150 to the bioprobe 110. While a rotation is one means of detachment, the specific motion used to detach the sheath 150 depends upon the specific means of attachment between the sheath 150 and bioprobe 110.

FIG. 11D depicts a schematic of the removal of the bioprobe 110 from the sheath removal system 840. When the bioprobe 110 is removed, the sheath 150 is retained by the sheath removal system 840 due to the engagement of the sheath release features 456 with the mating features 843 of the sheath removal system 840. When removed from the sheath removal system, the bioprobe body 116 will require engagement of a new sheath prior to use with a new patient.

After removal of the bioprobe 110, the sheath removal system 840 is used to crush or otherwise physically bend, cut, break, or otherwise physically deform the sheath 150. One method of crushing is depicted in FIG. 11E wherein a sheath crushing mechanism 846 is depressed into the sheath removal body 845, crushing the sheath 150. While the sheath is shown to be separated into multiple pieces, it may also be retained in a single piece by being bent, crushed or otherwise deformed through interaction of the sheath crushing mechanism 846 with the sheath 150. When the sheath is deformed, it is allowed to fall into a disposable medical waste bag 844 that is attached to the sheath removal body 845. One or more deformed sheaths 150 may be retained in the medical waste bag 844 prior to removal of the bag from the sheath removal body 845. The medical waste bag may be retained on the sheath removal body 845 through a variety of attachment means known to those skilled in the art, including retention of the bag by an adhesive means, through the use of clips, and equivalent means.

FIG. 12 depicts a flow chart that details one method of use of a BioProbe System including a bioprobe with sheath and sheath removal system, such as the sheath removal system depicted in FIGS. 11. The method of use includes attachment of a sterile sheath to the bioprobe, prior to use with a patient. The bioprobe with attached sheath is inserted into the body, or otherwise positioned so to collect data from the tissue under test. The bioprobe with attached sheath may be adjusted so to optimize the focus of a light-based image; this is preferably done using visible light, with the user adjusting the focus until the image appears in focus. Alternatively, an automated focus may be utilized wherein image quality criteria are used to automatically set the focus of the device through optimization of the criteria. When focus is achieved and light images or non-image data are collected in the visible portion of the spectrum, the visible light illuminator is switched off, with an appropriate illuminator turned on, so to excite the desired fluorescence from the tissue under test. The visible light and fluorescence data may be collected from one or more regions of tissue, so that the entire desired area for testing has been interrogated. When the entire desired area has been interrogated, the bioprobe is removed from the body or tissue under test. The bioprobe with attached sheath is next inserted into the sheath removal system with the sheath engaged in the sheath removal system reception port through one or more locking mechanisms. Successful engagement of the bioprobe and attached sheath in the sheath removal system is sensed through illumination of one or more detectors located in the feedback module. When the pulses of light from the one or more illuminators are sensed, the bioprobe system control module acknowledges detection of the pulses of light; if acknowledgement is not done, the pulses of light may be transmitted again, until the control module acknowledges receipt of these signals. When these pulsed signals are acknowledged, the control module releases the screening data so that it may be viewed on the display of the control module. When the screening data are released, the bioprobe may be removed from the sheath removal system. The sheath is deformed through depression of the sheath crushing apparatus; crushing of the sheath forces the deformed sheath into a medical waste bag. This medical waste bag may be removed and disposed as needed, with a new bag attached as needed. Preferably, the medical waste bag is attached to the sheath removal body with an interlocking feature so that the sheath removal system will not function without a medical waste bag in place. The sheath removal system may be cleaned as needed so to remove any biological contaminants.

FIGS. 13 represent another embodiment of a sheath removal system. In this embodiment, there is no sheath crushing mechanism, with the sheath falling into the medical waste bag as the bioprobe is detached from the sheath. The sheath removal system 940 comprises a sheath removal body 945 incorporating the receiving port 942 to receive the bioprobe with sheath, the sheath locking features (not shown), sheath removal feedback module 948, and disposable medical waste bag 944 attached to the sheath removal body 945. The bioprobe 110 having handle 111 and attached sheath 150, is inserted into the receiving port 942 where the sheath engages with the sheath locking features (not shown) of the sheath removal system 940. These sheath-locking features are similar to those described for the earlier embodiment of the sheath removal system as depicted in FIGS. 11.

Onto slide 953 are attached feedback module 948 and an optional deposition module 952. Feedback module 948 includes one or more optical detectors (not shown) that are used to confirm the presence of the bioprobe with sheath in the sheath removal system. The optional deposition module 952 comprises an assembly that applies a thin coating to the distal end portion of sheath 150. The distal end portion may be coated using a spray applicator, roller, pad, or other equivalent means to deposit a partially or completely opaque coating. The partially or completely opaque coating may be an ink, paint, a dye, plastic, or other material that will adhere to the optical window portion and render the sheath incapable of transmitting light as required by the BioProbe System.

FIG. 13A depicts a schematic of the sheath removal system 940 with reception port 942. Bioprobe 110 and attached sheath 150 are shown engaged in the reception port 942. It is assumed that the bioprobe with sheath has been used to collect images and/or related data from a single patient. While these images and/or related data may be viewed, the bioprobe system may not yet display screening information. Screening information includes identification of regions of human tissue where suspected abnormalities are located, the size of the suspected abnormal regions, the orientation of the abnormal regions with respect to surrounding tissues, the suspected degree of abnormality, and related information. Screening information is locked, and not made available to the person conducting the screening test, until the sheath removal system 940 acknowledges insertion and locking of the bioprobe 110 with sheath 150 into the reception port 942. The bioprobe 110 with sheath 150 is inserted into the sheath removal system 940 by positioning the distal end of the bioprobe with optical window 151 in the reception port 942. The bioprobe is pushed into the reception port until the sheath release features 956 engage with the mating features (not shown) of the sheath removal system 840. Engagement of release features 956 and mating features occurs when the bioprobe sheath 150 cannot be removed from the reception port 942. Alternatively, the system may display screening information and other data collected by the bioprobe, but will not release the data to storage and allow the bioprobe to be used with another patient until the sheath has been removed and replaced.

FIG. 13B shows the feedback module 948 positioned directly in line with the distal portion of the bioprobe and sheath. The feedback module 948 is attached to slide 953, which may be manually or automatically positioned in alignment with the bioprobe. Alignment is done so that light emitted by one or more of the bioprobe illuminators may be sensed by the feedback module 948. Prior to detachment of the sheath 150, one or more of the bioprobe illuminators (not shown) are pulsed on and off. The light emitted by the one or more illuminators is transmitted by the optical window 151 of the sheath 150, and impinges the feedback module 948 of the sheath removal system 940. The feedback module 948 includes one or more optical detectors and, optionally, one or more narrowband optical filters. The one or more narrowband optical filters are selected to transmit the light from the one or more bioprobe illuminators. When the pulses of light from the one or more illuminators are sensed, the bioprobe system control module acknowledges detection of the pulses of light; if acknowledgement is not done, the pulses of light may be transmitted again, until the control module acknowledges receipt of these signals. Pulsing of the one or more illumination signals in a predetermined manner allows the control module to differentiate these signals from ambient light. When these pulsed signals are acknowledged, the control module releases the screening data so that it may be viewed on the display. Full engagement of the sheath in the reception port 942 may be more rigorously determined through addition of an interlock that senses engagement of the sheath release features with the mating features of the sheath removal system 940.

FIG. 13C shows the optional deposition module 952 positioned directly in line with the distal portion of the bioprobe 110 and sheath 150. The deposition module 952 is attached to slide 953, which may be manually or automatically positioned in alignment with the bioprobe. Alignment is done so that the deposition module is located in close proximity to the optical window 151 of the sheath 150. The optional deposition module 952 comprises an assembly that applies a thin coating to the distal end portion of sheath 150, more specifically the optical window 151. The distal end portion may be coated using a spray applicator, roller, pad, or other equivalent means to deposit a partially or completely opaque coating. The partially or completely opaque coating may be an ink, paint, a dye, plastic, or other material that will adhere to the optical window portion and render the sheath incapable of transmitting light as required by the BioProbe System. Coating of the sheath window is optional and the deposition module may be eliminated from the sheath removal system if coating is not desired prior to sheath detachment from the bioprobe.

FIG. 13D depicts a schematic of detachment of the sheath 150 from the bioprobe 110. Detachment of the sheath may be done by a rotation of the bioprobe 110, through rotation of the bioprobe handle 111 relative to the reception port 942. This rotation is done to detach the features that join the sheath 150 to the bioprobe 110. While a rotation is one means of detachment, the specific motion used to detach the sheath 150 depends upon the specific means of attachment between the sheath 150 and bioprobe 110. Prior to detachment of the sheath 150, slide 953 is either manually or automatically moved so that both feedback module 948 and deposition module 952 are moved out of the path of the sheath as illustrated in FIG. 13D. When the bioprobe 110 is detached, the sheath 150 is free to fall into the waste disposal bag 944 that is attached to the sheath removal body 945. One or more used sheaths 150 may be retained in the medical waste bag 944 prior to removal of the bag from the sheath removal body 945. The medical waste bag may be retained on the sheath removal body 945 through a variety of attachment means known to those skilled in the art, including retention of the bag by an adhesive means, through the use of clips, and equivalent means. When removed from the sheath removal system, the bioprobe body 116 will require engagement of a new sheath prior to use with a new patient.

FIG. 14 depicts a flow chart that details a method of use of a bioprobe with sheath and sheath removal system 940 as illustrated in FIGS. 13. The method of use includes attachment of a sterile sheath to the bioprobe, prior to use with a patient. The bioprobe with attached sheath is inserted into the body, or otherwise positioned so to collect data from the tissue under test. The bioprobe with attached sheath may be adjusted so to optimize the focus of a light-based image; this is preferably done using visible light, with the user adjusting the focus until the image appears in focus. Alternatively, an automated focus may be utilized wherein image quality criteria are used to automatically set the focus of the device through optimization of the criteria. When focus is achieved and light images or non-image data are collected in the visible portion of the spectrum, the visible light illuminator is switched off, with an appropriate illuminator turned on, so to excite the desired fluorescence from the tissue under test. The visible light and fluorescence data may be collected from one or more regions of tissue, so that the entire desired area for testing has been interrogated. When the entire desired area has been interrogated, the bioprobe is removed from the body or the tissue under test. The bioprobe with attached sheath is next inserted into the sheath removal system with the sheath engaged in the sheath removal system reception port through one or more locking mechanisms. Successful engagement of the bioprobe and attached sheath in the sheath removal system is sensed through illumination of one or more detectors located in the feedback module. When the pulses of light from the one or more illuminators are sensed, the bioprobe system control module acknowledges detection of the pulses of light; if acknowledgement is not done, the pulses of light may be transmitted again, until the control module acknowledges receipt of these signals. When these pulsed signals are acknowledged, the control module releases the screening data so that it may be viewed on the display of the control module and saved to patient's records. Alternatively, the system may display screening information and other data collected by the bioprobe, but will not release the data to storage and allow the bioprobe to be used with another patient until the sheath has been removed and replaced. Following release of the screening data, the distal portion of the sheath may optionally be coated with an ink, paint, a dye, plastic, or other material that will adhere to the optical window portion and render the sheath incapable of transmitting light as required by the BioProbe System. The path between the sheath and medical waste bag is cleared following the release of the screening data and the optional coating of the distal portion of the sheath. As the bioprobe is removed from the sheath removal system, the sheath is released into the medical waste bag. This medical waste bag may be removed and disposed as needed, with a new bag attached as needed. Preferably, the medical waste bag is attached to the sheath removal body with an interlocking feature so that the sheath removal system will not function without a medical waste bag in place. The sheath removal system may be cleaned as needed so to remove any biological contaminants.

The invention has been described in detail with particular reference to certain preferred embodiments thereof, but it will be understood that variations and modifications can be effected within the spirit and scope of the invention.

What is claimed is:

1. A medical device comprising:
at least one bioprobe for collecting data;
a single-use protective cover mechanically attached to said at least one bioprobe;
a control module; and
a sheath removal system comprising:
a receiving port for said at least one bioprobe with said attached single-use protective cover;
a single-use protective cover locking mechanism for retaining said single-use protective cover; and
a single-use protective cover removal feedback mechanism in communication with said control module.

2. The medical device of claim 1 wherein said sheath removal system causes the collected data from said at least one bioprobe to be locked so that the user cannot access these data until the single-use protective cover has been captured and retained by the single-use protective cover locking mechanism of the sheath removal system.

3. The medical device of claim 1 wherein the sheath removal system causes the collected data from at least one bioprobe to be locked by the control module so that the user cannot use the bioprobe with a new patient until the single-use protective cover has been captured and retained by the single-use protective cover locking mechanism of the sheath removal system.

4. The medical device of claim 1 wherein said at least one bioprobe comprises at least one detector.

5. The medical device of claim 1 wherein said at least one bioprobe comprises an illuminator.

6. The medical device of claim 1 wherein said single-use protective cover is mechanically attached to said at least one bioprobe by a bayonet attachment.

7. The medical device of claim 1 wherein said the mechanical attachment of said single-use protective cover to said at least one bioprobe is adjustably positionable.

8. The medical device of claim 1 further comprising a disposal unit for said single-use protective cover after capture and retention by the sheath removal system.

9. A medical device comprising:
at least one bioprobe for collecting data;
a single-use protective cover mechanically attached to said at least one bioprobe;
a control module; and
a destructive sheath removal system comprising:
a receiving port for said at least one bioprobe with said attached single-use protective cover;
a single-use protective cover locking mechanism for retaining said single-use protective cover;
a single-use protective cover removal feedback mechanism in communication with said control module; and
a single-use protective cover destruction mechanism.

10. The medical device of claim 9 wherein said destructive sheath removal system causes the collected data from said at least one bioprobe to be locked so that the user cannot save these data until the single-use protective cover has been captured and retained by the single-use protective cover locking mechanism of the destructive sheath removal system.

11. The medical device of claim 9 wherein said at least one bioprobe comprises at least one detector.

12. The medical device of claim 9 wherein said at least one bioprobe comprises an illuminator.

13. The medical device of claim 9 wherein said single-use protective cover is mechanically attached to said at least one bioprobe by a bayonet attachment.

14. The medical device of claim 9 wherein said the mechanical attachment of said single-use protective cover to said at least one bioprobe is adjustably positionable.

15. The medical device of claim 9 wherein said destruction is a mechanical destruction selected from the group consisting of crushing, bending, cutting, breaking, or otherwise physically deforming said single-use protective cover.

16. The medical device of claim 9 wherein said destruction comprises rendering the sheath incapable of transmitting some or all of the illumination light.

17. The medical device of claim 9 further comprising a disposal unit for said single-use protective cover after destruction by said destructive sheath removal system.

18. A method comprising:
a). supplying at least one bioprobe for collecting data;
b). mechanically attaching a single-use protective cover said at least one bioprobe;
c). placing said at least one bioprobe with said mechanically attached single-use protective cover proximal a biological tissue to be interrogated;
d). removing said at least one bioprobe with said mechanically attached single-use protective cover from said biological tissue after collecting data;
e). placing said at least one bioprobe with said mechanically attached single-use protective cover into a sheath removal system comprising a receiving port for said at least one bioprobe with said attached single-use protective cover, a single-use protective cover locking mechanism for retaining said single-use protective cover, a single-use protective cover removal feedback mechanism in communication with said control module, and a medical waste container for retaining said single-use protective cover upon its removal from said bioprobe;
f). locking said at least one bioprobe with said mechanically attached single-use protective cover into said locking mechanism to trigger said single-use protective cover removal feedback mechanism in communication with a control module to release the collected data to the control module;
g). removing said at least one bioprobe for collecting data from the sheath removal system; and
h). retaining said single-use protective cover in said medical waste container.

19. A method comprising:
a). supplying at least one bioprobe for collecting data;
b). mechanically attaching a single-use protective cover said at least one bioprobe;
c). placing said at least one bioprobe with said mechanically attached single-use protective cover proximal a biological tissue to be interrogated;
d). removing said at least one bioprobe with said mechanically attached single-use protective cover from said biological tissue after collecting data;
e). placing said at least one bioprobe with said mechanically attached single-use protective cover into a sheath removal system comprising a receiving port for said at least one bioprobe with said attached single-use protective cover, a single-use protective cover locking mechanism for retaining said single-use protective cover, a single-use protective cover removal feedback mechanism in communication with said control module, a single-use protective cover destruction mechanism, and a medical waste container for retaining said single-use protective cover upon its removal from said bioprobe;
f). locking said at least one bioprobe with said mechanically attached single-use protective cover into said locking mechanism to trigger said single-use protective cover removal feedback mechanism in communication with a control module to release the collected data to the control module;
g). removing said at least one bioprobe for collecting data from the sheath removal system;
h). destroying the single-use protective cover retained in the locking mechanism with the destruction mechanism; and,
i). retaining said single-use protective cover in said medical waste container.
